(19) United States
(12) Patent Application Publication
(10) Pub. No.: US 2004/0097576 A1
(43) Pub. Date: May 20, 2004

(54) Use of tryptophan derivatives for the specific cytostatic treatment of serotonin-producing tumors

(76) Inventors: Diego Walther, Berlin (DE); Michael Bader, Berlin (DE)

Correspondence Address: DAVIDSON, DAVIDSON & KAPPEL, LLC, 485 SEVENTH AVENUE, 14TH FLOOR, NEW YORK, NY, 10018, US

(21) Appl. No.: 10/471,776

(22) PCT Filed: Mar. 15, 2002
(86) PCT No.: PCT/DE02/00959

(30) Foreign Application Priority Data
Mar. 15, 2001 (DE) 101 12 882.7

(51) Int. Cl. A61K 31/405

(52) U.S. Cl. 514/419

(57) ABSTRACT

The invention relates to the use of tryptophane-derivatives for the specific cytostatic treatment of serotonin-producing tumors, such as, for example carcinoids and the like. Areas of application of the invention are in the field of medicine and pharmaceutical industry.

### DESCRIPTION

The invention relates to the use of tryptophan-derivatives for specific cytostatic treatment of serotonin-producing tumors, such as, for example carcinoids and the like. Areas of application of the invention are in the field of medicine and pharmaceutical industry.

Carcinoids are unusual neuroendocrine tumors that mainly occur in the gastrointestinal tract and are developed by transformation of chromaffin cells in the Lieberkuhn's crypts (Neary et al., Dis. Colon Rectum 40: 349-362, 1997), and also exist in the lung and in the pancreas. Carcinoids can only be operatively removed, since conventional adjuvant therapy, in particular in advanced serotonin-producing tumors, is unsatisfactory, since these tumor cells are resistant against regular cytostatics (Neary et al., Dis. Colon Rectum 40: 349-362, 1997; Litvak et al., Surgery 124: 1071-1076, 1998). Thus, 3-18% of the patients die due to rapid metastasis of the primary tumor (Neary et al., Dis. Colon Rectum 40: 349-362, 1997; Memon and Nelson, Dis. Colon Rectum 40: 1101-1118, 1997).

Similarly, adverse prognoses are posed in a specific serotonin-producing lung tumor, the so-called small cell lung carcinoma, as well as in mastocytomas. In all serotonin-producing tumors, serotonin obviously functions as an autocrine growth-hormone, thus making it desirable, on the one hand, to reduce the serotonin-synthesis but, on the other hand, also to act on the tumor cells with specific toxins.

It is therefore an object of the present invention, to search for and to provide substances that enable the treatment of serotonin-producing tumors. These substances are supposed to, on one hand, function as specific cytostatics, at the same time they are also supposed to, as such, reduce the serotonin-synthesis.

The invention is put into practice according to the claims. It was found that the harmless substances 7-hydroxytryptophan and 6-hydroxytryptophan (7-HTP and 6-HTP, respectively) are metabolised intracellularly by the enzymes tryptophan-hydroxylase (TPH) and aromatic-amino acid-decarboxylase (AAAD) to the potent toxic substances 5,7- and 5,6-dihydroxytryptamine (5,7- and 5,6-DHT). These two enzymes, TPH and AAAD, are expressed extraordinarily strong in serotonin-producing tumor cells. Due to the unequally higher TPH-expression in these tumor cells in comparison with normal serotonin-producing tissues, only marginally toxic effects occur in these tissues.

According to the invention, therefore in particular hydroxylated tryptophan-derivatives that are, in particular, toxicated into specific toxins in the above-mentioned malignant cells by the enzymes tryptophan-hydroxylase (TPH) and aromatic-amino aciddecarboxylase (AAAD) of the body, are used for the reduction and/or inhibition of the synthesis of serotonin in tumor cells, whereby these cells are specifically killed. In contrast, other tissues maintain unaffected by the treatment.

Preferably, the tryptophan-derivatives 7-HTP, 6-HTP or 4-HTP, as well as further substituted derivatives at the aromatic system of tryptophan are used, which are metabolised into specific toxins. The toxins 5,7-DHT and 5,6-DHT, e.g., have been used in research for some time, in order to study lesions of tissues in which the serotonin-transporter is expressed. This leads also to a cytotoxic. effect in a multitude of serotonin-transporter-expressing cells that are not identical with serotonin-producing cells. In addition, these substances, due to their high sensitivity for oxidation, can be handled only with difficulties.

In addition, it was shown that the tryptophan-hydroxylase is competitively inhibited with hydroxylated tryptophan-derivatives since they compete with the actual substrate, tryptophan. This blocks the autocrine growth promotion of the serotonin in these malignant cells.

According to the invention, an effective cytostatic for the treatment of serotonin-producing tumors, such as, for example carcinoids using tryptophan-derivatives, in particular hydroxylated derivatives such as, for example, 7-HTP, 6-HTP or 4-HTP, is therefore provided. At the same time the tryptophan-derivatives selectively inhibit the tryptophane-hydroxylase, whereby a protection against the autocrine growth promotion of serotonin on the serotonin-producing tumors is achieved. Therefore, primary tumors can be treated, as well as metastases cytostatically be killed.

The cytostatic agents for the chemotherapy of malignant diseases on the basis of serotonin-producing tumors according to the invention are characterised in that they contain hydroxylated tryptophan-derivatives, preferably the derivatives 7-HTP, 6-HTP and 4-HTP, together with carriers and adjuvants known as such. In particular, they are used for therapy of neuroendocrine tumors that mainly occur in the gastrointestinal tract, in the lung and in the pancreas, and, in particular, for other serotonin-producing lung tumors, preferably the small cell lung carcinoma, as well as in mastocytoma.

For the direct application, these agents are preferably present in formulations for parenteral and/or oral application or, optionally, also in the form of liposomal complexes.

The invention shall be further explained by the following examples:

### EXAMPLES

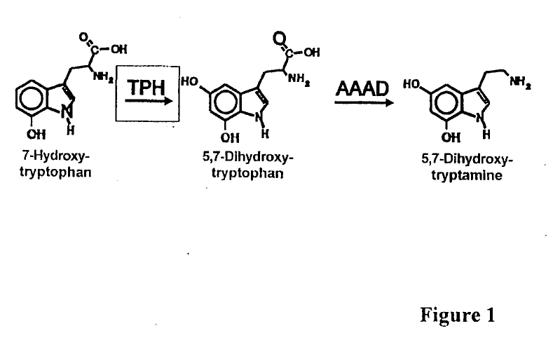

FIG. 1. Reaction scheme of enzymatic metabolisation of 7-hydroxytryptophan (7-HTP) to 5,7-dihydroxytryptamine (5,7-DHT). The rate-limiting first step is mediated by the enzyme tryptophan-hydroxylase (TPH) and is followed by a rapid decarboxylation by the aromatic-amino acid-decarboxylase (AAAD).

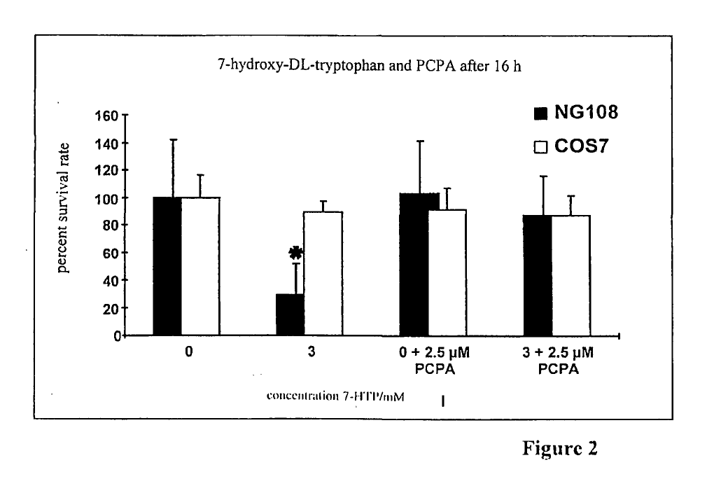

FIG. 2. Specific toxicity of 7-HTP on the serotonin-producing cells NG108. In contrast, the growth of COS7-cells that produce no serotonin is not affected by 7-HTP. The 7-HTP-sensitive NG 108-cells can be protected with the specific TPH-inhibitor p-chlorophenylalanin (PCPA), since no 5,7-DHT can be synthesised intracellularly.

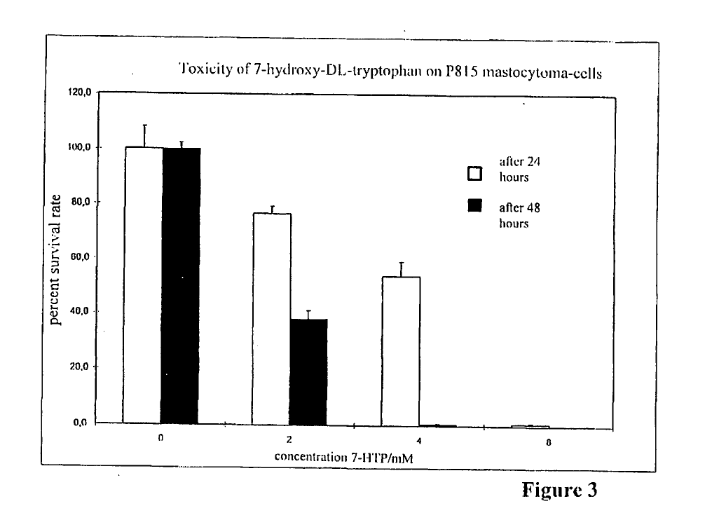

FIG. 3. Specific toxicity of 7-HTP on the serotonin-producing mastocytoma cells P815. These tumor cells that are characterised by a high resistance against regular cytostatics, are rapidly and specifically killed by 7-HTP.

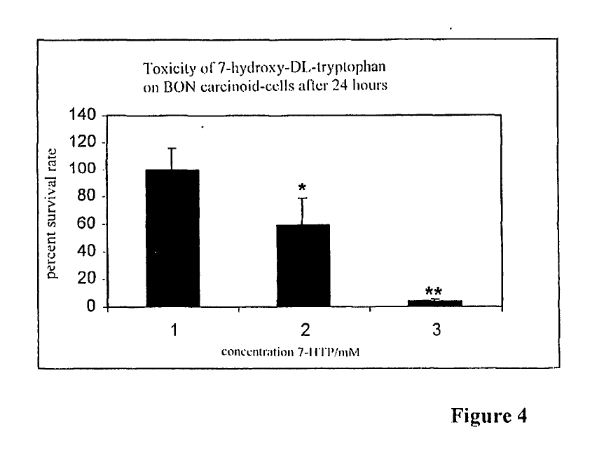

FIG. 4. Specific toxicity of 7-HTP on the serotonin-producing human carcinoid cells BON. These tumor cells that are characterised by a high resistance against common cytostatics, are rapidly and specifically killed by 7-HTP.

1. Use of tryptophane-derivatives, that are toxicated into specific toxins in a body's cell by the enzymes tryptophan-hydroxylase (TPH) and aromatic amino acid decarboxylase (AAAD) of the body, for the simultaneous reduction and/or disruption of the synthesis of serotonin in malignant serotonin-producing cells of the body.

2. Use according to claim 1, characterised in that the tryptophan-derivative, preferably hydroxylated tryptophane-derivatives, are used as cytostatics for serotonin-producing tumors and for metastasis that is caused by primary tumors.

3. Use of hydroxylated tryptophane-derivatives for the simultaneous competitive inhibition of tryptophan-hydroxylase (TPH) in serotonin-producing tumor cells.

4. Use according to claim 3, characterised in that the interaction of TPH and tryptophan is inhibited.

5. Use according to one of claims 1 to 4, characterised in that the tryptophan-derivatives 7-HTP, 6-HTP or 4-HTP, as well as derivatives that are further substituted at the aromatic system of tryptophan are used.

6. Use according to claim 5, characterised in that the tryptophan-derivatives 7-hydroxytryptophan and 6-hydroxytryptophan are metabolised into the specific toxins 5,7-dihydroxytryptamin (5,7-DHT) and 5,6-dihydroxytryptamin (5,6-DHT).

7. Cytostatic agent for therapy of malignant diseases, characterised in that it contains tryptophan-derivatives, preferably the hydroxylated derivatives 7-hydroxytryptophan, 6-hydroxytryptophan and 4-hydroxytryptophan, together with carriers and adjuvants, known as such.

8. Agent according to claim 7, characterised in that it is used for the therapy of neuroendocrine tumors that mainly occur in the gastrointestinal tract, in the lung and in the pancreas, and in certain other serotonin-producing lung tumors, preferably the small cell lung carcinoma, as well as in mastocytomas.
